(19) United States
(12) Patent Application Publication
(10) Pub. No.: US 2025/0060439 A1
(43) Pub. Date: Feb. 20, 2025

(54) MRI SYSTEM

(71) Applicant: CANON MEDICAL SYSTEMS CORPORATION, Otawara-shi (JP)

(72) Inventors: Takehiro SHIBUYA, Nasushiobara (JP); Sadanori TOMIHA, Nasushiobara (JP); Yoshitomo SAKAKURA, Nasushiobara (JP); Hiromi KAWAMOTO, Yaita (JP); Yuuzo KAMIGUCHI, Yokohama (JP); Michiru KAJIWARA, Nasushiobara (JP)

(73) Assignee: CANON MEDICAL SYSTEMS CORPORATION, Otawara-shi (JP)

(21) Appl. No.: 18/800,203

(22) Filed: Aug. 12, 2024

(30) Foreign Application Priority Data
Aug. 14, 2023 (JP) 2023-132149

(51) Int. Cl. G01R 33/421; A61B 5/055; G01R 33/34; G01R 33/3875

(52) U.S. Cl. CPC G01R 33/4215; G01R 33/34023; G01R 33/3875; A61B 5/055

(57) ABSTRACT

An MRI system includes a static magnetic field magnet configured to determine an MR frequency for imaging and generate a first static magnetic field be applied to an object during imaging; a pre-polarizing magnet configured to polarize nuclear spin of the object prior to imaging of the object and generate a second static magnetic field, application of which is stopped during imaging of the object; at least one first shield configured to block a leakage magnetic field of the first static magnetic field, the first shield being provided in a region opposite to an imaging region of the object with respect to the static magnetic field magnet; and at least one second shield configured to block a leakage magnetic field of the second static magnetic field, the second shield being provided in a region opposite to the imaging region with respect to the pre-polarizing magnet.

### CROSS-REFERENCE TO RELATED APPLICATION

This application is based upon and claims the benefit of priority from Japanese Patent Application No. 2023-132149, filed on Aug. 14, 2023, the entire contents of which are incorporated herein by reference.

### FIELD

Disclosed embodiments relate to a magnetic resonance imaging (MRI) system.

### BACKGROUND

An MRI apparatus is the main component of an MRI system and is an imaging apparatus that magnetically excites nuclear spin of an object placed in a static magnetic field by applying a radio frequency (RF) signal having the Larmor frequency and reconstructs an image on the basis of magnetic resonance (MR) signals emitted from the object due to the excitation.

Many MRI apparatuses have a configuration called a gantry in which a cylindrical space is formed. Imaging of an object (for example, a patient) lying on a table is performed in a state where the table is moved into the cylindrical space. Inside the gantry, a cylindrical superconducting magnet, a cylindrical gradient coil, and a cylindrical transmitting/receiving coil (i.e., WB (Whole Body) coil) are housed. Since the superconducting magnet, the gradient coil, and the transmitting/receiving coil are cylindrical, this structure adopted in many conventional MRI apparatuses is hereinafter referred to as a cylindrical MRI apparatus.

In the cylindrical MRI apparatus, imaging is performed in the cylindrical closed space, and thus, imaging may be difficult for some patients having claustrophobia, for example.

Accordingly, magnetic resonance imaging systems have been proposed and developed in a different manner that the superconducting magnets and gradient field coils respectively have a planar-shaped structure, and are configured to image a subject such as a patient in an open space between two planar superconducting magnets, for example. Hereinafter, an MRI apparatus having this type of structure is referred to as a planar open type MRI apparatus or simply referred to as an open type MRI apparatus. In the open type MRI apparatus, imaging is performed in the open space, and thus, even patients having claustrophobia can be imaged.

As compared with the static magnetic field magnet of the conventional cylindrical MRI apparatus, the static magnetic field magnets of the open type MRI apparatus tend to be placed closer to the floor and/or wall due to its shape. For example, when two planar superconducting magnets are placed horizontally, the superconducting magnet closer to the floor surface generates a strong magnetic field in the direction toward the floor surface. On the other hand, when two planar superconducting magnets are placed vertically, the superconducting magnet closer to the wall generates a strong magnetic field in the direction toward the wall.

These magnetic fields generated outside the superconducting magnets on the opposite side of the imaging space, i.e., leakage magnetic fields, can have negative influence on the space under the floor (for example, the space in the downstairs room) and the space behind the wall (for example, the space in the next room).

### BRIEF DESCRIPTION OF THE DRAWINGS

In the accompanying drawings:

FIG. 1 is a schematic diagram illustrating a first layout of magnet units used in an open type MRI apparatus that is the main component of an MRI system according to the first embodiment;

FIG. 2 is a schematic diagram illustrating a second layout of the magnet units used in the open type MRI apparatus;

FIG. 3 is a schematic perspective view of an internal configuration of the magnet units and illustrates a first layout of a magnetic shield (i.e., a first shield) and an electromagnetic shield (i.e., a second shield), both of which are components of the MRI system;

FIG. 4 is a schematic perspective view of an internal configuration of the magnet units and illustrates a second layout of the magnetic shield (i.e., the first shield) and the electromagnetic shield (i.e., the second shield), both of which are components of the MRI system;

FIG. 5 is a block diagram illustrating a configuration of the MRI apparatus included in the MRI system according to the first embodiment;

FIG. 6A to FIG. 6D are schematic diagrams illustrating a concept of a pulse sequence in which a pre-polarizing magnetic field B_{P} and a measurement magnetic field B₀ are applied in parallel with each other;

FIG. 7A to FIG. 7D are schematic diagrams illustrating a concept of a pulse sequence in which the pre-polarizing magnetic field B_{P} and the measurement magnetic field B₀ are applied perpendicularly to each other;

FIG. 8A and FIG. 8B are schematic diagrams illustrating a correction coil included in the second embodiment;

FIG. 9 is a block diagram illustrating a configuration of the MRI apparatus included in the MRI system according to the second embodiment;

FIG. 10 is a block diagram illustrating a concept of control of a correction current using a correction-coil control circuit; and

FIG. 11 is a schematic diagram illustrating a concept of controlling a pre-polarizing current and the correction current using the pre-polarizing control circuit.

### DETAILED DESCRIPTION

Hereinbelow, embodiments of the present invention will be described by referring to the accompanying drawings.

In one embodiment, an MRI system includes a static magnetic field magnet configured to determine a magnetic resonance frequency for imaging and generate a first static magnetic field to be applied to an object during imaging; a pre-polarizing magnet configured to polarize nuclear spin of the object prior to imaging of the object and generate a second static magnetic field, application of which is stopped during imaging of the object; at least one first shield configured to block a leakage magnetic field of the first static magnetic field, the at least one first shield being provided in a region opposite to an imaging region of the object with respect to the static magnetic field magnet; and at least one second shield configured to block a leakage magnetic field of the second static magnetic field, the at least one second shield being provided in a region opposite to the imaging region with respect to the pre-polarizing magnet.

### First Embodiment

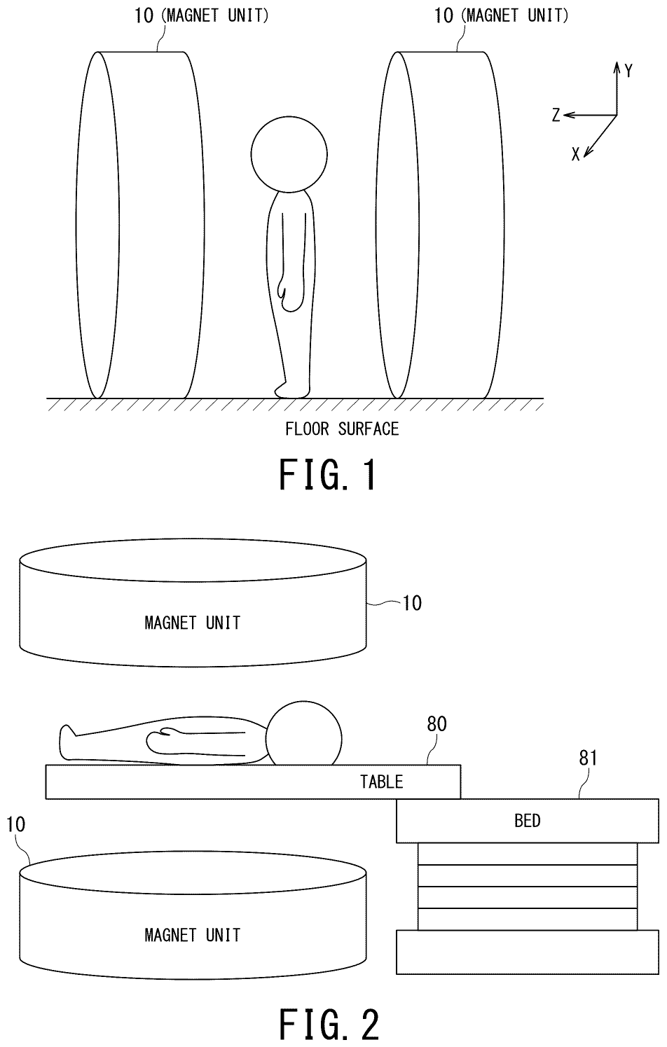

FIG. 1 illustrates a first layout of magnet units 10 used in an open type MRI apparatus 1 that is the main component of an MRI system 2 according to the first embodiment. As illustrated in FIG. 1, the MRI apparatus 1 includes two magnet units 10, each of which has a circular flat planar shape (i.e., a thin cylindrical shape), for example.

Each magnet unit 10 is arranged such that the central axis (i.e., the axis passing through the centers of both circular end faces of the cylindrical shape) is parallel to the floor surface, for example. Further, the two magnet units 10 are arranged so as to sandwich the object. Such an arrangement generates a magnetic field in the open space between the two magnet units 10. The object is imaged in this open space in a standing position, for example.

FIG. 2 illustrates a second layout of the magnet units 10 that are used in the open type MRI apparatus 1. While FIG. 1 illustrates an arrangement for imaging an object in a standing position, FIG. 2 illustrates an arrangement for imaging an object in a recumbent position lying on a table 80 of a bed 81. When the object in the recumbent position is imaged, the two magnet units 10 are arranged such that their central axes are in the vertical direction as shown in FIG. 2. For example, one of the magnet units 10 is disposed below the table 80 and the other of the magnet units 10 is disposed above the table 80.

As shown in FIG. 1 and FIG. 2, in the imaging using the magnet units 10 of the planar structure, the object can be imaged in an open magnetic field space, and thus, even a patient with claustrophobia can be imaged, for example.

Although FIG. 1 and FIG. 2 illustrate a case where the whole object is imaged in an open space outside the magnet units 10 containing superconducting coils, it can be part of the object placed and imaged in the open space outside the superconducting coils.

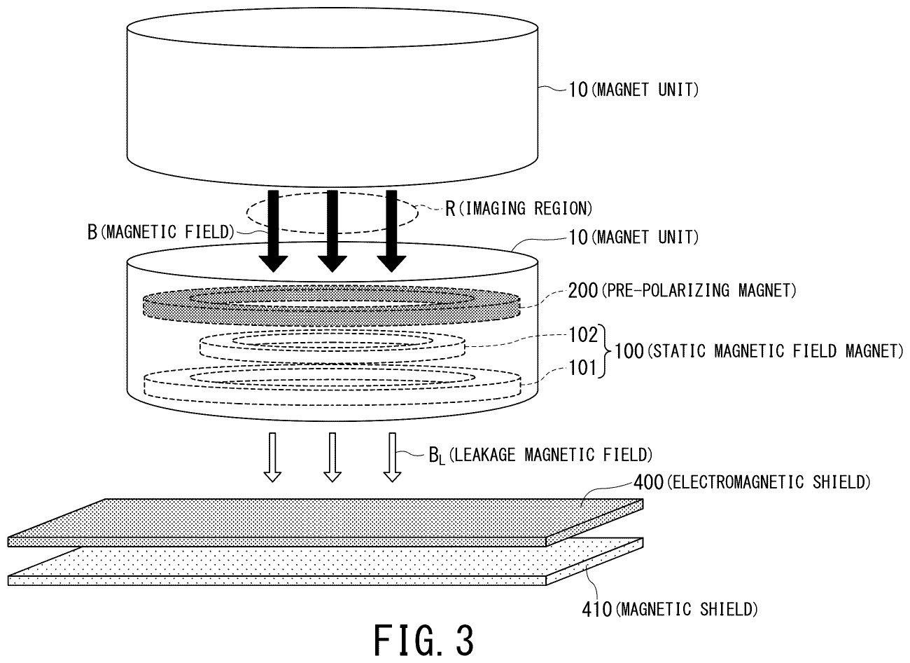

FIG. 3 is a schematic perspective view of the internal configuration of the magnet units 10 and illustrates a first layout of a magnetic shield 410 (i.e., at least one first shield) and an electromagnetic shield 400 (i.e., at least one second shield), both of which are components of the MRI system 2.

As shown in FIG. 3, each magnet unit 10 includes a static magnetic field magnet 100 and a pre-polarizing magnet 200. The static magnetic field magnet 100 determines the magnetic resonance frequency for imaging and generates a first static magnetic field that is applied to the object during imaging. Although the static magnetic field magnet 100 can be composed of one magnet, the static magnetic field magnet 100 may be composed of a first magnet 101 configured to generate a magnetic field in a forward direction and a second magnet 102 configured to generate a magnetic field in a reverse direction opposite to the forward direction, as illustrated in FIG. 3.

The pre-polarizing magnet 200 polarizes nuclear spin of the object prior to imaging of the object, and generates a second static magnetic field, application of which is stopped during imaging of the object. The configuration and functions of the static magnetic field magnet 100 and the pre-polarizing magnet 200 will be described below in detail.

FIG. 3 is a schematic perspective view corresponding to the second layout of the magnet units 10, and the two magnet units 10 are vertically arranged with an imaging space R interposed therebetween. The magnet units 10 generate a vertical magnetic field B in the imaging space R as shown by the filled thick arrows in FIG. 3.

A leakage magnetic field BL is also generated in the region opposite to the imaging space R with respect to the lower magnet unit 10, as shown by the blank thin arrows in FIG. 3. This leakage magnetic field BL does not contribute to imaging of the object in the imaging space R.

The magnetic fields generated in the direction opposite to the magnetic field in the imaging space, i.e., the leakage magnetic field BL can have negative influence on the space under the floor (for example, the space in the downstairs room) and the space behind the wall (for example, the space in the next room).

In the second layout of the magnet units 10, the lower magnet unit 10 is disposed close to the floor surface, and thus, generates a strong leakage magnetic field BL in the direction toward the floor surface.

For this reason, in the MRI system 2 according to the first embodiment, two different shields are provided to block or suppress the leakage magnetic field BL. The first shield is the magnetic shield 410 for blocking or suppressing non-dynamic (i.e., static) magnetic fields (i.e., magnetic fields with constant strength), and the second shield is the electromagnetic shield 400 for blocking or suppressing dynamic magnetic fields.

The second shield may be a shield coil for blocking or suppressing a pulsed magnetic field, for example. The first shield may be configured as a magnetic shield that also serves as the second shield.

As described below, the second static magnetic field (i.e., the pre-polarizing magnetic field) generated by the pre-polarizing magnet 200 is turned on and turned off with a steep rise and a steep fall by controlling the electric current applied to the pre-polarizing magnet 200. Thus, the second static magnetic field is a dynamic static magnetic field and has electromagnetic properties. Hence, the electromagnetic shield 400 is suitable for the second static magnetic field.

The electromagnetic shield 400 is made of highly conductive materials such as a galvanized steel sheet, a steel plate, and an aluminum plate. On the other hand, the magnetic shield 410 is made of materials with high magnetic permeability, such as a permalloy, an electromagnetic pure iron, a silicon steel plate, and amorphous. The electromagnetic shield 400 and the magnetic shield 410 can be installed in the members constituting the floor, for example.

In the first layout in which the respective magnet units 10 are vertically arranged, the magnet unit 10 closer to the wall generates a strong magnetic field in the direction toward the wall. In this case, the magnetic shield 410 (i.e., the first shield) and the electromagnetic shield 400 (i.e., the second shield) can be embedded in the wall nearby.

In this manner, the leakage magnetic field BL can be efficiently suppressed by installing the electromagnetic shield 400 and the magnetic shield 410 in the region opposite to the imaging region of the object with respect to the magnet units 10.

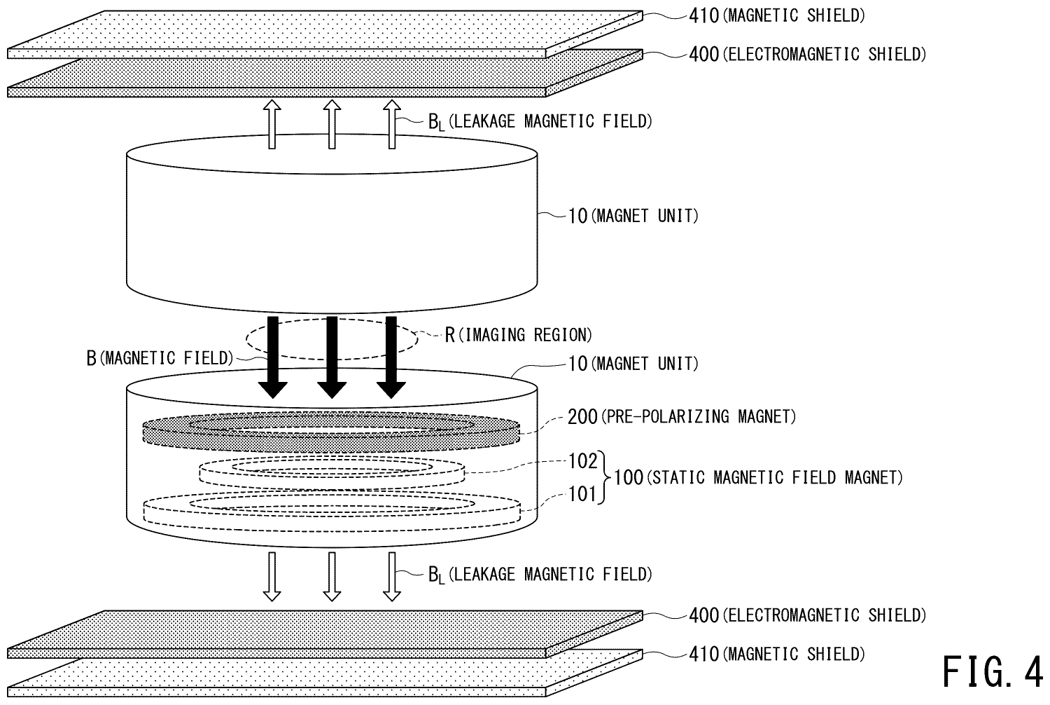

FIG. 4 is a schematic perspective view illustrating a second layout of the magnetic shield 410 (i.e., the first shield) and the electromagnetic shield 400 (i.e., the second shield). In the layout in which the two magnet units 10 are disposed above and below the lying object P, the leakage magnetic field BL may also be generated by the upper magnet unit 10.

In such a case, as illustrated in FIG. 4, it is satisfactory if the electromagnetic shield 400 and the magnetic shield 410 are installed not only in the members constituting the floor but also in the members constituting the ceiling. Such a configuration and layout of the electromagnetic shield 400 and the magnetic shield 410 can suppress the influence of the leakage magnetic field BL not only on the downstairs room but also on the upstairs room.

It should be noted that in each magnet unit 10 in FIGS. 3 and 4, the arrangement of the first magnet 101, the second magnet 102, and the pre-polarizing magnet 200 is not limited to the order shown in FIGS. 3 and 4. For example, in FIGS. 3 and 4, in the lower magnet unit 10, the pre-polarizing magnet 200, the second magnet 102, and, the first magnet 101, are arranged in this order from the top in the figure, but this is not limited to this and they can be arranged in any order.

In addition, the number of each of the first magnets 101, second magnets 10, and pre-polarizing magnets 200 in each magnet unit 10 is not limited to one, and the number of each of the magnets 101, second magnets 10, and pre-polarized magnets 200 can also be two or more.

Furthermore, the arrangement of the electromagnetic shield 400 (second shield) and the magnetic shield 410 (first shield) is not limited to the order shown in FIGS. 3 and 4. For example, the electromagnetic shield 400 and the magnetic shield 410 may be arranged in the reverse order to that shown in FIGS. 3 and 4.

Furthermore, the number of each of the upper and lower electromagnetic wave shields 400 and magnetic shields 410 is not limited to one but may be two or more.

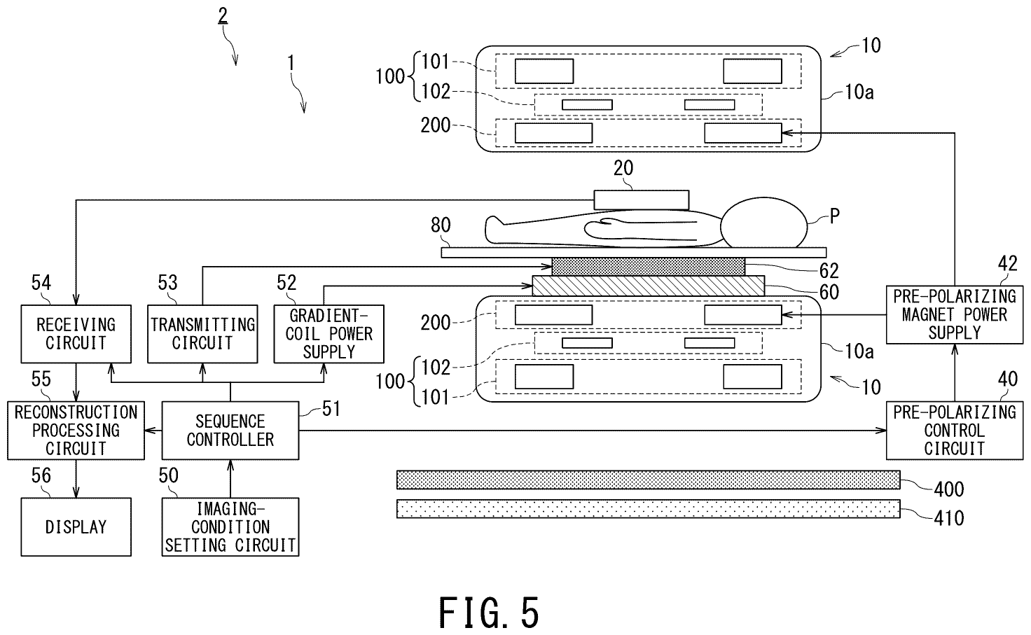

FIG. 5 is a block diagram illustrating a configuration of the MRI apparatus 1 included in the MRI system 2 according to the first embodiment. FIG. 5 illustrates a configuration of the MRI apparatus 1 having the second layout in which the above-described magnet units 10 are disposed above and below the lying object P. An imaging region or FOV (Field of View) is formed between the two magnet units 10.

The MRI apparatus 1 shown in FIG. 5 includes: the table 80 on which the object P lies, a local coil 20 installed close to the object P, a gradient coil 60, and an RF coil 62 in addition to the two magnet units 10.

The MRI apparatus 1 further includes an imaging-condition setting circuit 50, a sequence controller 51, a gradient-coil power supply 52, a transmitting circuit 53, a receiving circuit 54, a reconstruction processing circuit 55, and a display 56.

The imaging-condition setting circuit 50 sets or selects imaging conditions, such as a type of pulse sequence and values of various parameters inputted via a user interface (not shown), on the sequence controller 51.

The sequence controller 51 performs a scan of the object by driving the gradient-coil power supply 52 and the transmitting circuit 53 based on the selected imaging conditions.

The gradient-coil power supply 52 applies gradient magnetic field currents to the gradient coil 60 based on the drive signal from the sequence controller 51.

The transmitting circuit 53 generates an RF pulse based on the drive signal from the sequence controller 51, and applies the RF pulse to the RF coil 62. Each MR (Magnetic Resonance) signal emitted from the object P in response to application of the RF pulse is received by the local coil 20. The MR signals received by the local coil 20 are converted from analog signals into digital signals by the receiving circuit 54. The digitized MR signals are inputted as k-space data to the reconstruction processing circuit 55. The reconstruction processing circuit 55 performs reconstruction processing such as inverse Fourier transform on the k-space data to generate MR images. The generated MR images are displayed on the display 56.

As described above, each of the magnet units 10 includes the pre-polarizing magnet 200, and the static magnetic field magnet 100 which is composed of the first magnet 101 and the second magnet 102. The static magnetic field magnet 100 and the pre-polarizing magnet 200 are configured as planar superconducting magnets, for example.

Although the static magnetic field magnet 100 and the pre-polarizing magnet 200 may be configured as planar superconducting magnets as described above, the embodiments are not limited to such an aspect. For example, the first magnet 101 and the second magnet 102 of the static magnetic field magnet 100 may be configured as superconducting magnets, while the pre-polarizing magnet 200 may be configured as a normal conducting magnet (i.e., an electromagnet that operates at a room temperature). Conversely, the first magnet 101 and the second magnet 102 of the static magnetic field magnet 100 may be configured as normal conducting magnets, while the pre-polarizing magnet 200 may be configured as a superconducting magnet. Alternatively, all of the pre-polarizing magnet 200 and the first and second magnets 101 and 102 of the static magnetic field magnet 100 may be configured as normal conducting magnets.

The static magnetic field magnet 100 and the pre-polarizing magnet 200 are housed in a container 10a. When both the static magnetic field magnet 100 and the pre-polarizing magnet 200 are configured as superconducting magnets, both are housed in a cooling container 10a called a cryostat, for example.

As shown in FIG. 5, the MRI apparatus 1 includes a pre-polarizing magnet power supply 42 configured to apply an electric current to the pre-polarizing magnet 200 and a pre-polarizing control circuit 40 configured to control the pre-polarizing magnet power supply 42. As described below, the MRI apparatus 1 is configured to be able to execute a pulse sequence in which the pre-polarizing magnetic field (i.e., the second static magnetic field) is used. The pre-polarizing magnet 200, the pre-polarizing magnet power supply 42, and the pre-polarizing control circuit 40 are provided for generating the pre-polarizing magnetic field.

As described above, electric currents flow through the respective coils of the first magnet 101 and the second magnet 102 in such a manner that the direction of the magnetic field generated by the second magnet 102 is opposite to the direction of the magnetic field generated by the first magnet 101. Thus, when the sign representing the magnetic field generated by the first magnet 101 is positive, the sign representing the magnetic field generated by the second magnet 102 is negative.

As a result, in the combined magnetic field, the magnetic field generated by the first magnet 101 and the magnetic field generated by the second magnet 102 cancel each other out, and a region with approximately uniform magnetic field strength (i.e., a region where magnetic field strength hardly changes) can be generated in the cylindrical axis direction, i.e., in the up-and-down direction of the sheet of each of FIG. 2 to FIG. 5. The FOV during MR imaging can be set inside such a region where the magnetic field strength hardly changes, i.e., a region where approximately uniform magnetic field strength B0 in the cylindrical axis direction (i.e., Y-axis direction) is available.

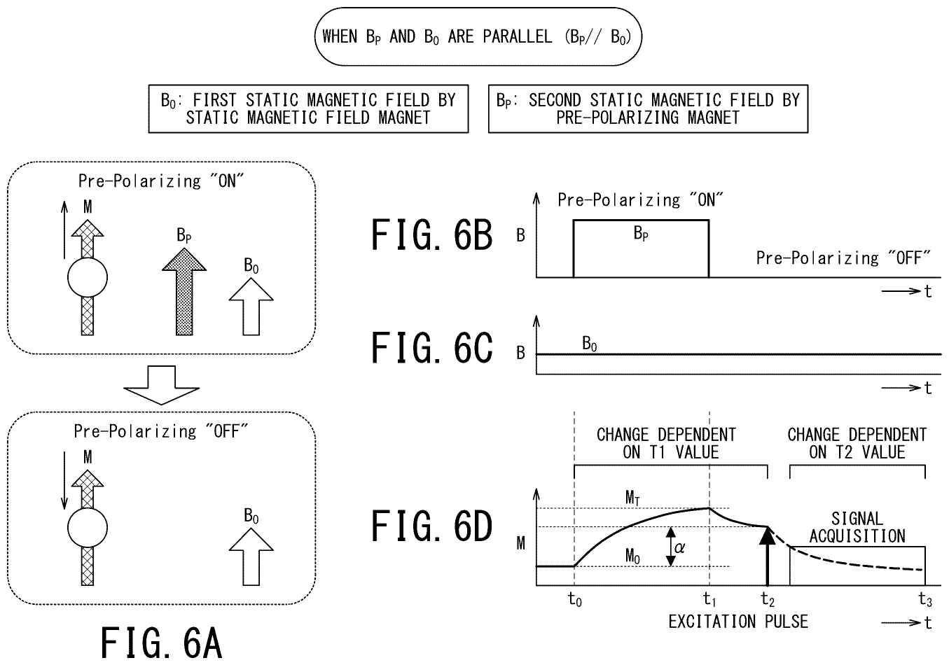

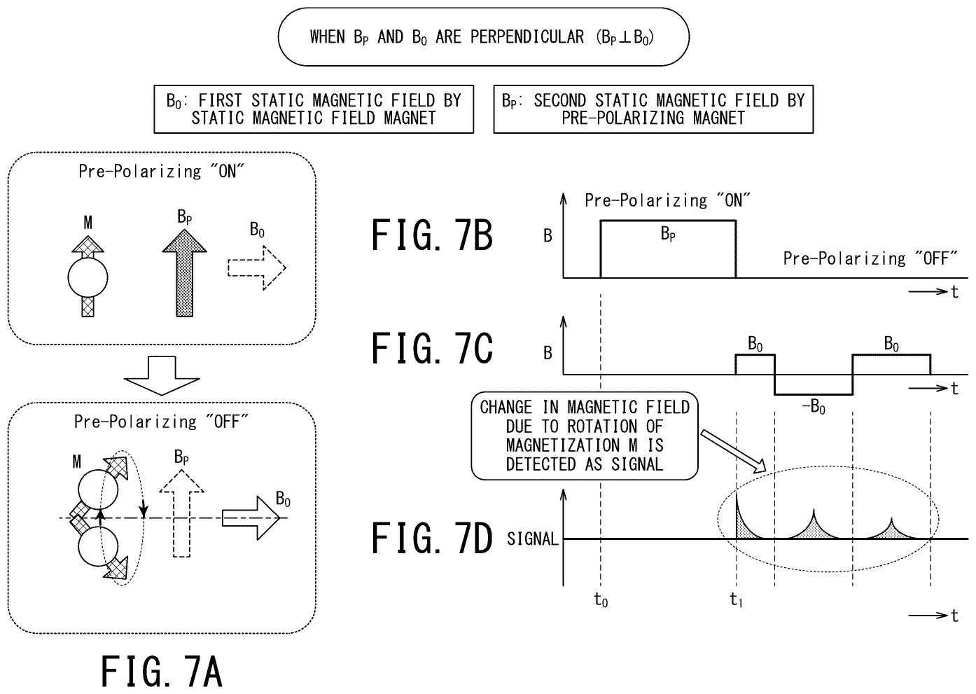

FIG. 6A to FIG. 6D illustrate a pulse sequence in which the pre-polarizing magnetic field (i.e., the second static magnetic field) is used. FIG. 7A to FIG. 7D illustrate another pulse sequence in which the pre-polarizing magnetic field is used as well. Each of these two pulse sequences uses two static magnetic fields composed of the first static magnetic field B₀ and the pre-polarizing magnetic field BP (i.e., the second static magnetic field BP). The first static magnetic field B₀ determines the magnetic resonance frequency for imaging and is applied to the object during imaging. Meanwhile, the pre-polarizing magnetic field B_{P} polarizes nuclear spin of the object prior to imaging of the object, and application of the pre-polarizing magnetic field B_{P} is stopped during imaging of the object. Since the object is imaged or measured under the first static magnetic field B₀, the first static magnetic field B₀ may be hereinafter referred to as the measurement magnetic field B₀ as appropriate.

The pulse sequence using the pre-polarizing magnetic field B_{P} includes at least two application methods as follows. One method is to have both the pre-polarizing magnetic field B_{P} and the measurement magnetic field B₀ applied in parallel with each other, and the other is to have both applied perpendicularly to each other.

FIG. 6A to FIG. 6D illustrate a concept of the pulse sequence in which the pre-polarizing magnetic field B_{P} and the measurement magnetic field B₀ are applied in parallel with each other. As shown in FIG. 6A to FIG. 6D, the pre-polarizing magnetic field is applied prior to imaging of the object. For example, as shown in FIG. 6D, the pre-polarizing magnetic field B_{P} is turned on at a time to and turned off at a time t₁. Thereafter, at a time t₂, an excitation pulse is applied to start imaging of the object, and the MR signal of the object is acquired.

The measurement magnetic field B₀ is the combined magnetic field of the magnetic fields respectively generated by the first magnet 101 and the second magnet 102, as shown in FIG. 6C. This measurement magnetic field B₀ is constantly and continuously applied to the object before the pre-polarizing magnetic field B_{P} is turned on, as shown in FIG. 6C.

In the period during which the pre-polarizing magnetic field B_{P} is turned on, the pre-polarizing magnetic field B_{P} and the measurement magnetic field B₀ are applied in the same direction, so the combined magnetic field applied to the object is formed by adding pre-polarizing magnetic field B_{P} to the measurement magnetic field B₀. As a result, the magnetization M (or spin M) generated in the object is enhanced by the pre-polarizing magnetic field BP.

FIG. 6D schematically illustrates temporal change in behavior of the magnetization M of the object. Before application of the pre-polarizing magnetic field B_{P}, the magnetization M of the object is the magnetization M₀ generated by the measurement magnetic field B₀ only. However, after application of the pre-polarizing magnetic field B_{P}, the magnetization M of the object is enhanced to the magnetization M_{T} by an exponential function dependent on a T1 value (i.e., longitudinal relaxation time), which is a tissue parameter of the object. At the time t₁ at which the pre-polarizing magnetic field is turned off, the magnetization M begins to decrease. At the same time, the magnetization M decreases by an exponential function dependent on the T1 value (i.e., longitudinal relaxation time). Subsequently, at the time t₂, the excitation pulse is applied, and the longitudinal magnetization M becomes transverse magnetization. Thereafter, though the transverse magnetization decreases by an exponential function dependent on a T2 value (i.e., longitudinal relaxation time) which is a tissue parameter, the transverse magnetization is detected or acquired as an MR signal during this decreasing period, and the acquired MR signals are used to generate an MR image.

In the above-described pulse sequence in which the pre-polarizing magnetic field B_{P} is applied, the magnetization M of the object can be increased by increasing the strength of the pre-polarizing magnetic field B_{P} applied immediately before imaging, while lowering the strength of the constantly generated measurement magnetic field B₀. In such manner, an MR image with a high signal-to-noise ratio (SNR) can be generated, even with the lower measurement magnetic field B₀.

Since the strength of the measurement magnetic field B₀ constantly applied is maintained low, the leakage magnetic fields can be suppressed except for the short period during which the pre-polarizing magnetic field B_{P} is applied.

FIG. 7A to FIG. 7D illustrate a concept of the pulse sequence in which the pre-polarizing magnetic field B_{P} and the measurement magnetic field B₀ are applied perpendicularly to each other. In this pulse sequence, the pre-polarizing magnetic field B_{P} is applied prior to imaging of the object as well.

For example, as shown in FIGS. 7B to 7D, the pre-polarizing magnetic field B_{P} is turned on at the time to and is turned off at the time t₁. Thereafter, as shown in FIG. 7C, the measurement magnetic field B₀ is applied, for example, three times while changing its sign. Application of such a measurement magnetic field B₀ generates echoes similarly to the GRE (gradient echo) method, and MR signals can be acquired.

In the pulse sequence shown in FIG. 7A to FIG. 7D, the direction of the magnetization M generated by the pre-polarizing magnetic field B_{P} and the direction of measurement magnetic field B₀ are perpendicular to each other. Thus, after the pre-polarizing magnetic field B_{P} is turned off, the magnetization M approaches the measurement magnetic field B₀ while rotating around the measurement magnetic field B₀. In the pulse sequence shown in FIG. 7A to FIG. 7D, change in the magnetic field due to the rotation of the magnetization M is detected as each MR signal.

In the pulse sequence in which the pre-polarizing magnetic field B_{P} and the measurement magnetic field B₀ are applied perpendicularly to each other as shown in FIG. 7A to FIG. 7D, the magnetization M of the object can also be enhanced by increasing the strength of the pre-polarizing magnetic field B_{P} applied immediately before imaging. As a result, an MR image with a high signal-to-noise ratio (SNR) can be generated.

The measurement magnetic field B₀ only needs to be applied for a short period during which MR signals are acquired, and the strength of the measurement magnetic field B₀ can also be kept low. Consequently, in the pulse sequence shown in FIG. 7A to FIG. 7D, the leakage magnetic fields can also be suppressed except for the short period during which the pre-polarizing magnetic field B_{P} is applied.

According to the MRI system 2 of the first embodiment described above, because the magnetic shield 410 (i.e., the at least one first shield) and the electromagnetic shield 400 (i.e., the at least one second shield) are provided in the region opposite to the imaging region of the object with respect to the magnet units 10, and a pulse sequence with application of the pre-polarizing magnetic field B_{P} is used, the leakage magnetic fields can be suppressed.

### Second Embodiment

The MRI system 2 of the second embodiment differs from the first embodiment in that the MRI system 2 of the second embodiment has a correction coil 300 for correcting a magnetic field and a configuration for controlling an electric current to be applied to the correction coil 300, while the rest of the configurations of the second embodiment are the same as the first embodiment.

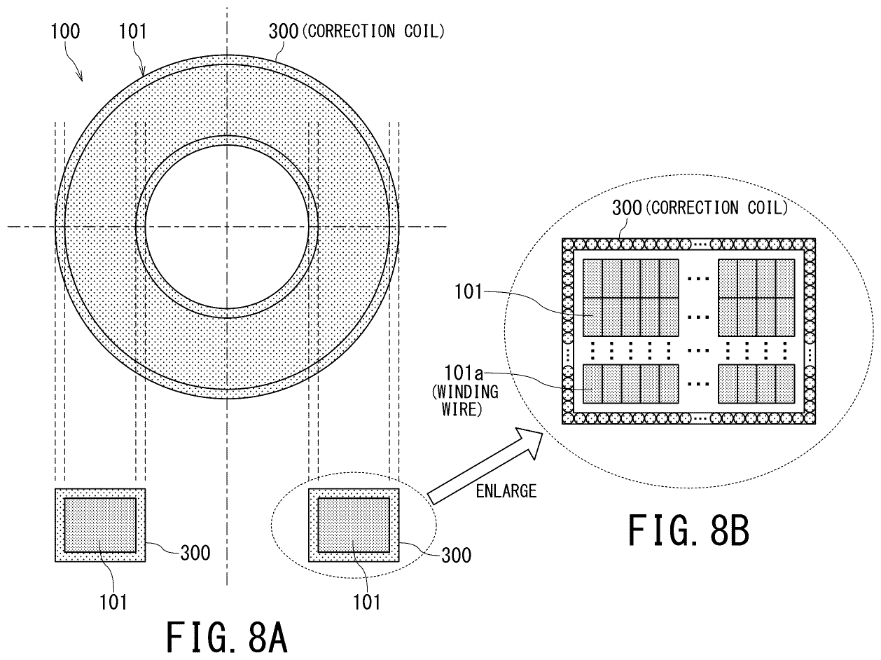

FIG. 8A and FIG. 8B illustrate the correction coil 300 included in the second embodiment. For example, the correction coil 300 can be provided so as to surround part or all of the outer surface of at least one of the first magnet 101 and the second magnet 102. Applying a correction current to the correction coil 300 can correct the static magnetic field distribution of at least one of the first static magnetic field (i.e., the measurement magnetic field B₀), the second static magnetic field (i.e., the pre-polarizing magnetic field B_{P}), and the combined static magnetic field of the first and second static magnetic fields.

FIG. 8A and FIG. 8B illustrate one aspect of the correction coil 300. However, the correction coil 300 is not limited to the aspect shown in FIG. 8A and FIG. 8B. The correction coil 300 can be in various forms as long as it can correct the static magnetic field distribution of at least one of the first static magnetic field (i.e., the measurement magnetic field B₀), the second static magnetic field (i.e., the pre-polarizing magnetic field B_{P}), and the combined static magnetic field of the first and second static magnetic fields.

The correction coil 300 may be formed using metallic wire materials, such as copper wire, aluminum wire, nichrome wire. Alternatively, the correction coil 300 may be formed using superconducting wire materials, such as NbTi, Nb₃Sn, MgB₂, Bi-based wire materials and REBCO wire materials.

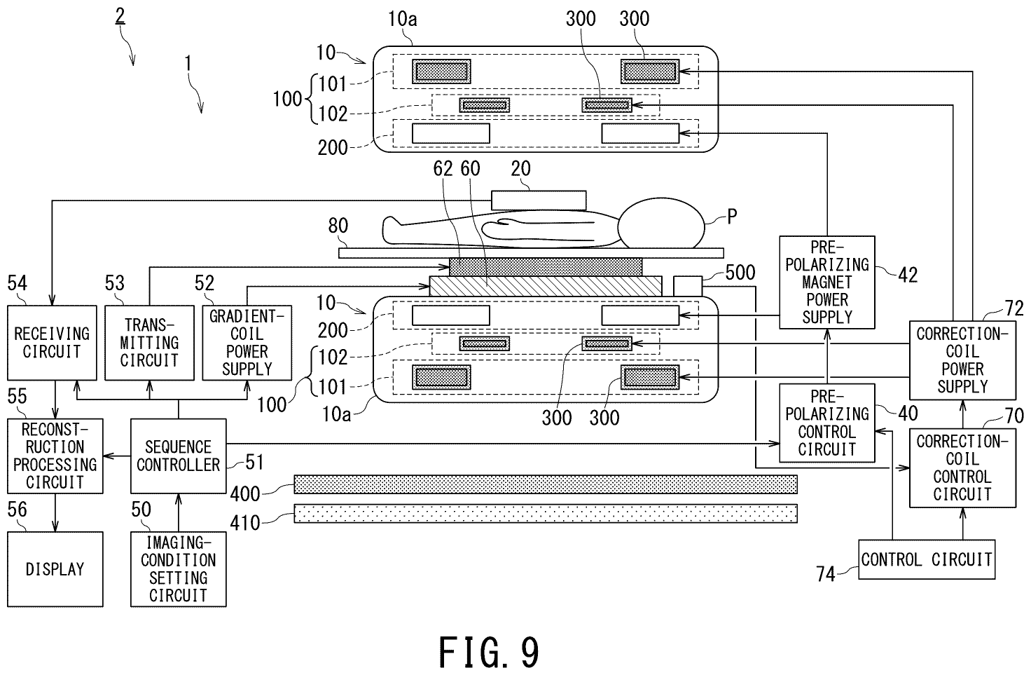

FIG. 9 is a block diagram illustrating a configuration of the MRI apparatus 1 included in the MRI system 2 according to the second embodiment. The difference from the first embodiment (FIG. 4) is that the MRI apparatus 1 in the second embodiment further includes the correction coil 300. The MRI system 2 of the second embodiment includes at least one magnetic field sensor 500, a control circuit 74, a correction-coil control circuit 70, and a correction-coil power supply 72 in order to control the correction current to be applied to the correction coil 300.

The magnetic field sensor 500 measures at least one of the first static magnetic field (i.e., the measurement magnetic field B₀), the second static magnetic field (i.e., the pre-polarizing magnetic field B_{P}), and the combined static magnetic field of the first and second static magnetic fields.

The magnet unit 10 includes the container 10a that houses the static magnetic field magnet 100, the pre-polarizing magnet 200, and the correction coil 300. The magnetic field sensor 500 is provided outside the container 10a, for example. The number of the magnetic field sensors 500 may be one or plural.

The correction-coil power supply 72 supplies the correction current to the correction coil 300 based on the magnetic field measured by at least one magnetic field sensor 500 such that the static magnetic field distribution of at least one of the first static magnetic field (i.e., the measurement magnetic field B₀), the second static magnetic field (i.e., the pre-polarizing magnetic field B_{P}), and the combined static magnetic field of the first and second static magnetic fields is corrected.

The correction-coil control circuit 70 controls the correction current to be supplied from the correction-coil power supply 72 to the correction coil 300.

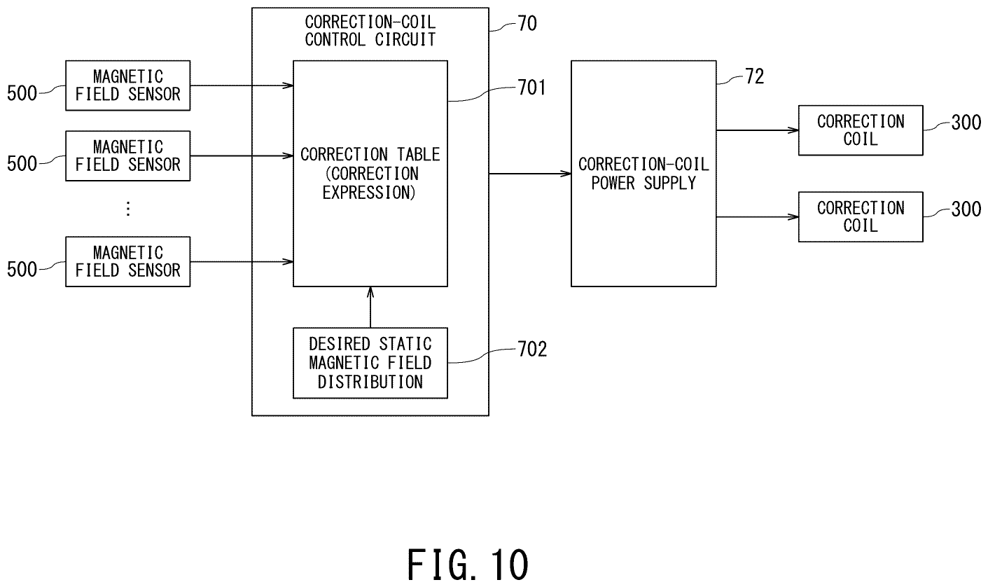

FIG. 10 is a block diagram illustrating a concept of control of the correction current using the correction-coil control circuit 70. The correction-coil control circuit 70 stores a correction table 701 (or correction expression 701), as shown in FIG. 10. The static magnetic field distribution of at least one of the first static magnetic field (i.e., the measurement magnetic field B₀), the second static magnetic field (i.e., the pre-polarizing magnetic field B_{P}), and combined static magnetic field of the first and second static magnetic fields is under the influence of the magnetic shield 410 (i.e., the first shield) and the electromagnetic shield 400 (i.e., the second shield). Because of such influence, changes such as offset may occur, which may cause errors in desired static magnetic field distribution.

Thus, the correction-coil control circuit 70 feedback-controls the correction current to be supplied to the correction coil 300 from the correction-coil power supply 72 based on the magnetic field measured by at least one magnetic field sensor 500 and the predetermined correction table 701 (or correction expression 701), such that the static magnetic field distribution of at least one of the first static magnetic field (i.e., the measurement magnetic field B₀), the second static magnetic field (i.e., the pre-polarizing magnetic field B_{P}), and the combined static magnetic field of the first and second static magnetic fields matches the desired static magnetic field distribution.

### Modification of Second Embodiment

As shown in FIG. 6D, in the pulse sequence in which the pre-polarizing magnetic field B_{P} is applied, the value of the magnetization M changes depending on the tissue parameters of the object, such as a T1 value (i.e., longitudinal magnetization relaxation time), a T2 value, and a T2* value (i.e., transverse magnetization relaxation time or apparent transverse magnetization relaxation time). Further, the value of the magnetization M also changes depending on the sequence parameters. For example, the value of the magnetization M changes depending on the sequence parameters such as an application time length of the pre-polarizing magnetic field B_{P}, a time length from stopping application of the pre-polarizing magnetic field B_{P} to application of the excitation pulse, a time length from application of the excitation pulse to the start of acquisition of MR signals, and an elapsed time from stopping application of the pre-polarizing magnetic field B_{P} to the start of imaging.

Thus, in order to obtain desired MR signals during imaging, it is preferred to appropriately control the magnitude of the longitudinal magnetization and/or the transverse magnetization. Accordingly, it is preferred that the pre-polarizing current to be applied to the pre-polarizing magnet 200 and the correction current to be applied to the correction coil 300 are controlled based on the tissue parameters of the object to be imaged and the sequence parameters of the pulse sequence to be used for imaging the object.

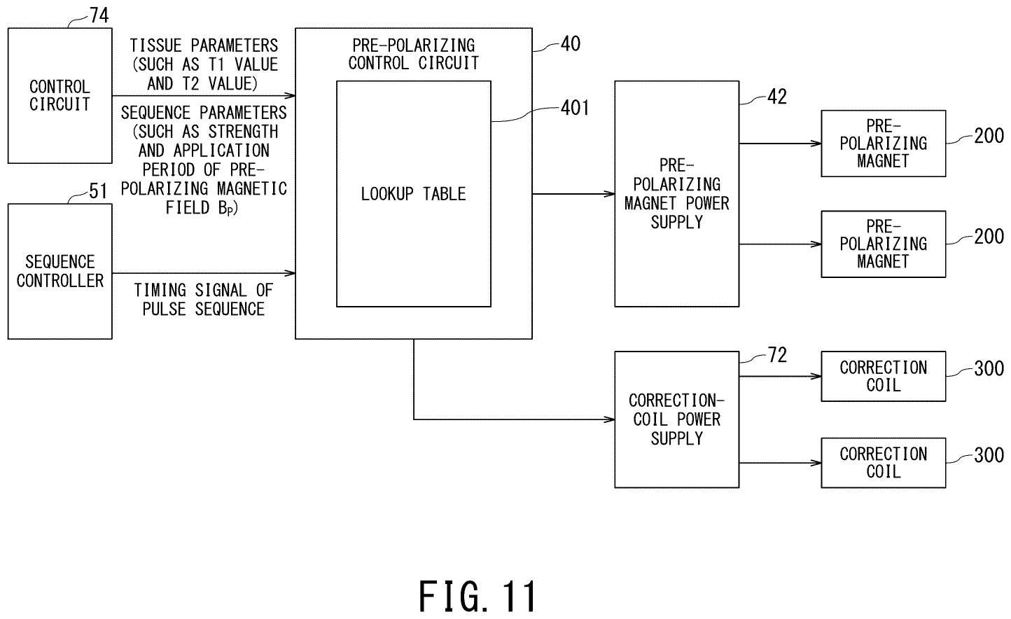

Accordingly, in the modification of the second embodiment as shown in FIG. 11, the pre-polarizing control circuit 40 acquires the tissue parameters, such as the T1 value and the T2 value, and the sequence parameters, such as the desired strength value of the pre-polarizing magnetic field B_{P} and the desired application time length, from the control circuit 74, and acquires a timing signal of the pulse sequence from the sequence controller 51.

On the basis of the acquired tissue parameters and sequence parameters and the acquired timing signal of the pulse sequence, the pre-polarizing control circuit 40 controls parameters related to at least one of the correction current and the pre-polarizing current (such as a current value, an on/off timing of the current, a rise time, and a fall time) by, for example, referring to an internally stored lookup table 401.

Under such control, desired MR signals can be acquired in the pulse sequence in which the pre-polarizing magnetic field B_{P} is applied, and consequently, a desired MR image can be generated.

The MRI apparatus 1 of each of the above-described embodiments (for example, FIG. 2 to FIG. 5, and FIG. 9) has a configuration in which two magnet units 10 are provided to face each other and the object is imaged in the space between both magnet units 10.

However, the embodiments are not limited to the above-described aspect, and the MRI apparatus 1 may be configured with only one of both magnet units 10. For example, the MRI apparatus 1 of each embodiment may be configured by having the magnet unit 10 above the table 80 removed from the MRI apparatus 1 shown in FIG. 5 or FIG. 9. Even in such a configuration, each of the above-described technical effects can be achieved.

According to at least one embodiment described above, leakage magnetic fields generated in the open-type MRI apparatus can be suppressed.

While certain embodiments have been described, these embodiments have been presented by way of example only, and are not intended to limit the scope of the invention. Indeed, the novel methods and systems described herein may be embodied in a variety of other forms; furthermore, various omissions, substitutions and changes in the form of the methods and systems described herein may be made without departing from the spirit of the invention. The accompanying claims and their equivalents are intended to cover such forms or modifications as would fall within the scope and spirit of the invention.

What is claimed is:

1. An MRI system comprising:
a static magnetic field magnet configured to determine a magnetic resonance frequency for imaging and generate a first static magnetic field to be applied to an object during imaging;
a pre-polarizing magnet configured to polarize nuclear spin of the object prior to imaging of the object and generate a second static magnetic field, application of which is stopped during imaging of the object;
at least one first shield configured to block a leakage magnetic field of the first static magnetic field, the at least one first shield being provided in a region opposite to an imaging region of the object with respect to the static magnetic field magnet; and
at least one second shield configured to block a leakage magnetic field of the second static magnetic field, the at least one second shield being provided in a region opposite to the imaging region with respect to the pre-polarizing magnet.

2. The MRI system according to claim 1, wherein:
the at least one first shield is further provided in a region in a same direction as the imaging region with respect to the static magnetic field magnet; and
the at least one second shields is further provided in a region in a same direction as the imaging region with respect to the pre-polarizing magnet.

3. The MRI system according to claim 1,
wherein, the at least one first shield is configured as a magnetic shield, and
the at least one second shield is configured as an electromagnetic shield.

4. The MRI system according to claim 1,
wherein, the at least one first shield is configured as a magnetic shield, and
the at least one second shield is configured as a coil.

5. The MRI system according to claim 1,
wherein the at least one first shield is configured as a magnetic shield that also serves as the at least one second shield.

6. The MRI system according to claim 1,
wherein the static magnetic field magnet is configured as a planar superconducting magnet.

7. The MRI system according to claim 6,
wherein the planar superconducting magnet includes:
at least one first magnet configured to generate a magnetic field in a forward direction, and
at least one second magnet configure to generate a magnetic field in a direction opposite to the forward direction.

8. The MRI system according to claim 1,
wherein a direction of the second static magnetic field generated by the pre-polarizing magnet is perpendicular to a direction of the first static magnetic field generated by the static magnetic field magnet.

9. The MRI system according to claim 1,
wherein a direction of the second static magnetic field generated by the pre-polarizing magnet is parallel to a direction of the first static magnetic field generated by the static magnetic field magnet.

10. The MRI system according to claim 1, further comprising:
a correction coil configured to correct a static magnetic field distribution of at least one of the first static magnetic field, the second static magnetic field, and a combined static magnetic field that is a combination of the first static magnetic field and the second static magnetic field;
at least one magnetic field sensor configured to measure at least one of the first static magnetic field, the second static magnetic field, and the combined static magnetic field; and
a correction-coil power supply configured to supply an electric current to the correction coil depending on a magnetic field measured by the at least one magnetic field sensor in such a manner that the static magnetic field distribution of at least one of the first static magnetic field, the second static magnetic field, and the combined static magnetic field is corrected.

11. The MRI system according to claim 10, further comprising a container configured to house the static magnetic field magnet, the pre-polarizing magnet, and the correction coil,
wherein the magnetic field sensor is provided outside a space surrounded by the container.

12. The MRI system according to claim 10,
wherein the static magnetic field distribution of at least one of the first static magnetic field, the second static magnetic field, and the combined static magnetic field is affected by the at least one first shield and the at least one second shield.

13. The MRI system according to claim 10, further comprising a correction-coil control circuit configured to control an electric current from the correction-coil power supply,
wherein the correction-coil control circuit feedback-controls the electric current from the correction-coil power supply, based on the magnetic field measured by the at least one magnetic field sensor and a predetermined correction table or a predetermined correction expression, in such a manner that the static magnetic field distribution of at least one of the first static magnetic field, the second static magnetic field, and the combined static magnetic field matches a desired static magnetic field distribution.

14. The MRI system according to claim 1, further comprising:
a correction coil configured to correct a static magnetic field distribution of at least one of the first static magnetic field, the second static magnetic field, and a combined static magnetic field that is a combination of the first static magnetic field and the second static magnetic field;
a correction-coil power supply configured to supply a correction current to the correction coil;
a pre-polarizing magnet power supply configured to supply a pre-polarizing current to a pre-polarizing coil that constitutes the pre-polarizing magnet; and
a pre-polarizing control circuit configured to control the correction current and the pre-polarizing current based on a tissue parameter of the object to be imaged and a sequence parameter of a pulse sequence to be used for imaging the object.

15. The MRI system according to claim 14, wherein the pre-polarizing control circuit is configured to control a parameter related to at least one of the correction current and the pre-polarizing current based on the tissue parameter and the sequence parameter.

16. The MRI system according to claim 14, wherein:
the tissue parameter includes a longitudinal relaxation time (T1 value) of a tissue in the object; and
the sequence parameter includes at least one of an application time of the second static magnetic field to be generated by the pre-polarizing magnet and an elapsed time from a time of stopping application of the second static magnetic field to a start of imaging of the object.
